(19) United States
(12) Patent Application Publication
(10) Pub. No.: US 2025/0169918 A1
(43) Pub. Date: May 29, 2025

(54) METHODS AND SYSTEMS FOR A CONNECTION DEVICE

(71) Applicant: GE Precision Healthcare LLC, Waukesha, WI (US)

(72) Inventors: Paul Krueger, Pewaukee, WI (US); Stephen G Hushek, Wauwatosa, WI (US); Brian Kohr, Hartland, WI (US); Jason L Philps, Merton, WI (US); Joshua T Anders, Oconomowoc, WI (US)

(21) Appl. No.: 18/522,069

(22) Filed: Nov. 28, 2023

(51) Int. Cl. A61B 90/57

(52) U.S. Cl. CPC A61B 90/57; A61B 2090/571

(57) ABSTRACT

Systems are provided for a connection device. In one example, a system includes a first device, a second device, and a connection device coupled to the first device via a pin and to the second device via a clamp. A tether extends from the pin to the clamp and is configured to flex and maintain a position of the connection device.

### FIELD

Embodiments of the subject matter disclosed herein relate to a connection device, particularly to a connection device for devices in a medical setting.

### BACKGROUND

Hospitals, medical offices, and other healthcare settings are stressed due to a lack of staff and an increasing number of patients. Healthcare workers may be expected to perform tasks individually that were previously performed by multiple people. Additionally, healthcare workers may be expected to work more quickly in order to complete various tasks and improve patient health outcomes.

### BRIEF DESCRIPTION

In one example, a system includes a first device, a second device, and a connection device coupled to a coupling element mounted on the first device at a first end and clamped to the second device at a second end, wherein the connection device further comprises a tether extending from the first end to the second end.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 is an example of a connection device coupling a first device to a second device;

FIG. 2A is an embodiment of the connection device;

FIG. 2B is a cross-section of a tether of the connection device;

FIG. 3 is a pin end of the connection device engaging with a pin received of the first device;

FIG. 4 is an embodiment of the connection device clamped to the second device and coupled to the first device;

FIG. 5A is an embodiment of the connection device in a stored position on the first device;

FIG. 5B is an embodiment of the connection device extending from a first side of the first device; and

FIG. 5C is an embodiment of the connection device extending from a second side of the first device.

### DETAILED DESCRIPTION

The following description relates to various embodiments for a connection device. The connection device comprises a tether comprising a brush clamp at a first extreme end and a pin at a second extreme end, wherein the pin engages with a pin hole of a first device and the brush clamp clamps to a tube of a second device. The pin hole may be arranged on a quick connect device mounted to the first device. The quick connect may be one of a plurality of quick connect devices mounted at different locations of the first device.

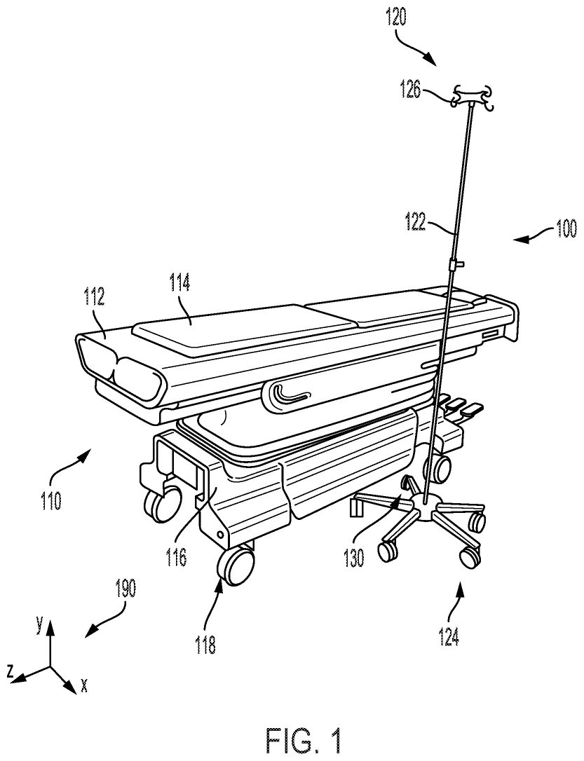

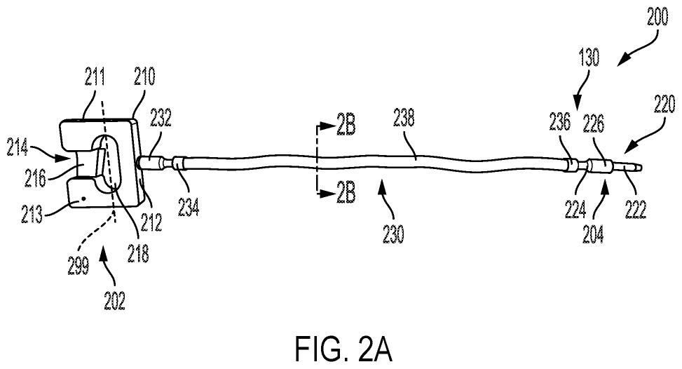

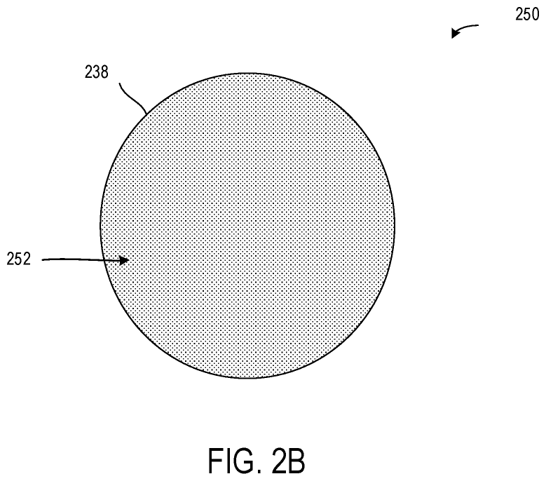

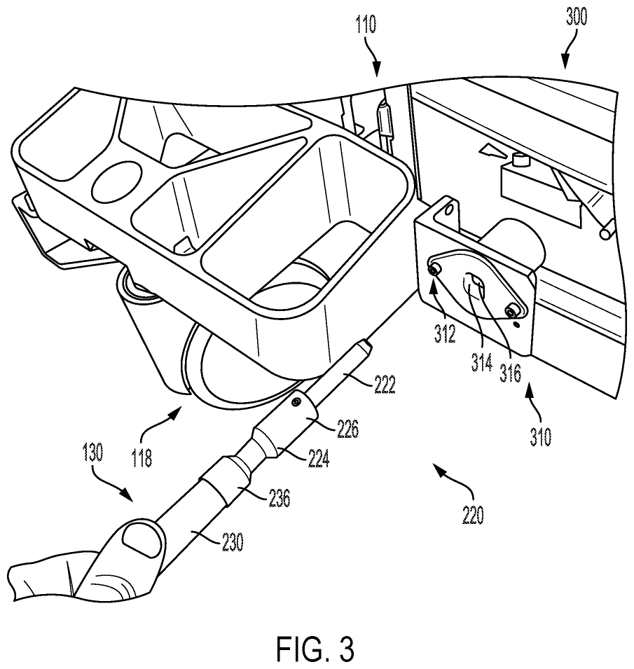

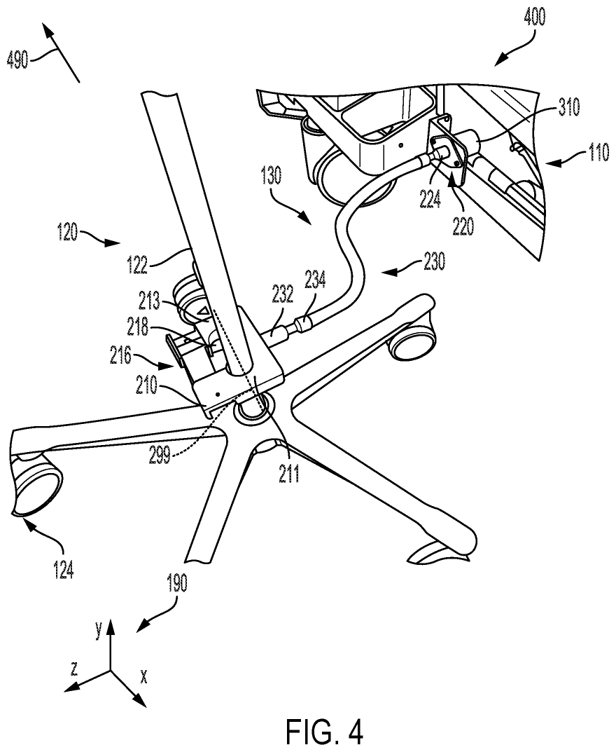

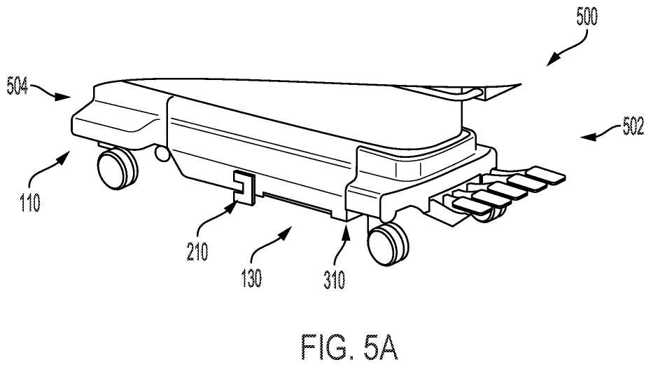

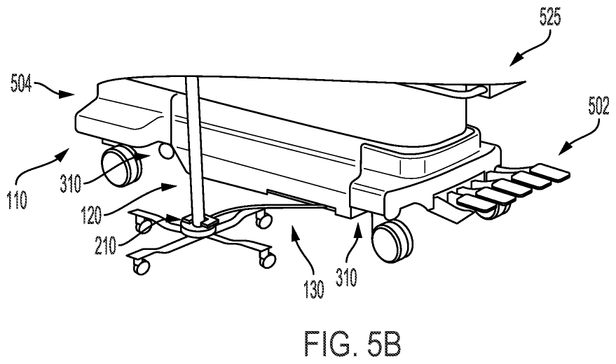

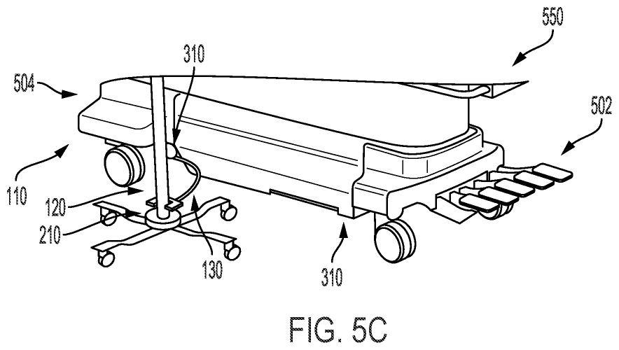

FIG. 1 is an example of a connection device coupling a first device to a second device. FIG. 2A is an embodiment of the connection device. FIG. 2B is a cross-section of a tether of the connection device. FIG. 3 is a pin end of the connection device engaging with a pin coupler of the first device. FIG. 4 is an embodiment of the connection device clamped to the second device and coupled to the first device. FIG. 5A is an embodiment of the connection device is a stored position on the first device. FIG. 5B is an embodiment of the connection device extending from a first side of the first device. FIG. 5C is an embodiment of the connection device extending from a second side of the first device.

Turning now to FIG. 1, it shows an embodiment 100 of a first device 110, a second device 120, and a connection device 130. An axis system 190 is shown comprising three axes, namely an x-axis parallel to a lateral direction, a y-axis parallel to a vertical direction, and a z-axis parallel to a longitudinal direction. A direction of gravity may be opposite a positive direction of the y-axis.

The first device 110 may include a platform 112 coupled to a body 116. A pad 114 may be arranged on the platform 112. A patient may lay on the pad 114 while a healthcare worker pushes the first device 110 through a medical environment. A plurality of wheels 118 may be coupled to the body 116. In one example, the platform 112 is arranged on a top of the body 116 and the plurality of wheels 118 are arranged on a bottom of the body 116, wherein the bottom is opposite to the top relative to the direction of gravity. In one example, the first device 110 is a bed cart.

A second device 120 may include a body 122. In one example, the body 122 is a tube. A plurality of wheels 124 may be coupled to a bottom of the body 122. A plurality of hooks 126 may be coupled to a top of the body 122, wherein the top is opposite to the bottom. In one example, the plurality of hooks 126 may couple to a bag, such as an intravenous fluid (IV) bag. In one example, the second device 120 is a rolling IV pole.

A connection device 130 may be coupled to the first device 110 at a first end and to the second device 120 at a second end. The connection device 130 may transfer energy from the first device 110 to the second device 120. As such, when the first device 110 is pushed and rolled across a ground on which the first device 110 is arranged, the second device 120 may move in conjunction with the first device 110. In one example, the connection device 130 may extend in a direction angled to the direction of gravity. For example, the connection device 130 may extend normal to gravity when coupled to the first device 110 and the second device 120.

Turning now to FIG. 2A, it shows an embodiment 200 of the connection device 130. The connection device 130 may include a first end 202 and a second end 204. The first end 202 may be opposite the second end 204.

A clamp body 210 may be arranged at the first end. The clamp body 210 may include a U-shape having a first arm 211 and a second arm 213 extending from a body 212. In one example, the clamp body 210 is symmetric. The clamp body 210 may be a brush clamp.

The first arm 211 may extend in a direction normal to the body 212 and the second arm 213 may extend in the direction normal to the body 212. The first arm 211 and the second arm 213 may be identical to one another in size and shape. The first arm 211 and the second arm 213 may include a “d” shape. Said another way, the first arm 211 and the second arm 213 may include a first portion having a first width that extends from the body 212 and a second portion with a second width extending from the first portion. The second width is greater than the first width. In one example, the first arm 211 and the second arm 213 comprise a curved, asymmetrical shape.

The first arm 211 and the second arm 213 may be spaced apart from one another by an opening 214 and a slot 218. A plurality of bristles 216 may extend across the opening 214 and control entry and exit of a device into the slot 218, such as the second device 120 of FIG. 1. The plurality of bristles 216 may be physically coupled to one of the first arm 211 or the second arm 213 and extend toward the opposite arm. In one example, the plurality of bristles 216 are physically coupled to the second arm 213 and extend toward and touch the first arm 211 across an entire distance of the opening 214.

The plurality of bristles 216 may include a thickness and density configured to maintain the second device 120 within the slot 218 when the first device 110 is being rolled while also allowing a healthcare worker to quickly and easily force the second device 120 passed the plurality of bristles 216 and into the slot 218. Each bristle may include a diameter between 0.2 to 0.4 mm. Additionally or alternatively, a diameter of each bristle may be between 0.3 to 0.4 mm. In one example, the diameter of each bristle is 0.36 mm.

A density of the plurality of bristles 216 may include between 150 to 250 bristles per cm, or 175 to 225 bristles per cm, or 190 to 210 bristles per cm. In one example, the plurality of bristles 216 includes 200 bristles per cm.

The slot 218 may be larger than the opening 214. In one example, a distance of the slot 218, measured along its longitudinal axis 299, extends beyond a distance of the opening 214 measured along an axis parallel to the longitudinal axis 299. By doing this, the second device 120 may be arranged at an extreme end of the slot 218 and misaligned with the opening 214. In this way, the first arm 211 and/or the second arm 213, along with the plurality of bristles 216, may retain the second device 120 within the slot 218.

The second end 204 may include a pin 220. The pin 220 may include a first body 222 and a second body 224. The first body 222 may include a cylindrical shape with a first diameter. The second body 224 may include a cylindrical shape with a second diameter, wherein the second diameter greater than the first diameter.

A third body 226 may be arranged between the first body 222 and the second body 224. The third body 226 may include linear sections in combination with curved sections. In one example, the third body 226 comprises a square shape with curved corners. Additionally or alternatively, the third body 226 may include a square with 90° corners.

An arm 230 may be coupled to each of the clamp body 210 and the pin 220. The arm 230 may include a first connector 232 coupling a first cap 234 of the arm 230 to the clamp 210. In one example, the first connector 232 is physically coupled to the body 212 via a fastener, wherein the fastener is concealed via the first connector 232. The fastener may be accessible from the slot 218.

A second cap 236 may be arranged at the second end 204. In one example, the second cap 236 is integrally formed with the second body 224. The first cap 234 and the second cap 236 may extend over an outer layer 238 of the arm 230. The first cap 234 and the second cap 236 may be fixedly coupled to the arm 230 via one or more of an adhesive, a weld, a fusion, and a compression fit.

The arm 230 may be flexible. The arm 230 may include a single wire surrounded by the outer layer 238, wherein the wire is configured to bend, twist, kink, and execute other actions that deviate from linear. A length of the arm 230 may be fixed. In one example, the arm 230 may be rigid along its longitudinal axis normal to the axis 299 and flexible (e.g., adjustable) along its short axis, normal to the longitudinal axis and parallel to a radius of the arm 230.

Turning now to FIG. 2B, it shows a cross-section 250 of the arm 230 taken along cutting plane 2B of FIG. 2A. In the example of FIG. 2B, the outer layer 238 may surround a plurality of stranded wires 252. In one example, the outer layer 238 is a single, continuous layer comprising nylon or another material. The plurality of stranded wires 252 may be copper, aluminum, or another material. In this way, the arm 230 may include a single wire or a plurality of wires.

In one example, the arm 230 is a tether, a rope, or other similar device. A diameter of the arm 230 may be between 5 to 10 mm, 6 to 9 mm, or 7 to 8 mm. In one example, the diameter of the arm 230 is equal to exactly 7.3 mm and comprises a 1 gauge (e.g., 1 American wire gauge (AWG)).

Turning now to FIG. 3, it shows an embodiment 300 of the pin 220 being directed toward a coupling element 310. Herein, the coupling element 310 is referred to as a quick connect 310. The quick connect 310 may be physically coupled to the first device 110 via a plurality of fasteners 312. The plurality of fasteners 312 may thread into mounting through-holes. The mounting through-holes may be positioned closer to the plurality of wheels 118 than to a platform (e.g., platform 112 of FIG. 1). By mounting the quick connect 310 to a lower region, a stability of the second device coupled to the first device 110 via the connection device 130 may be increased relative to a higher mounting point.

Additionally or alternatively, the quick connect 310 may be coupled to the first device 110 via welds, fusions, adhesives, tape, and other similar elements. The first device 110 may include a plurality of mounting points, each configured to receive and maintain the quick connect 310 in a fixed position.

The quick connect 310 may include a first opening 314 and a second opening 316. The first opening 314 may be larger than the second opening 316. In one example, the first body 222 may engage with surfaces shaping the second opening 316 and the third body 226 may engage with surfaces shaping the first opening 314. As such, the first opening 314 may include linear portions that complementarily fit with linear portions of the third body 226. The first body 222 may be retained in the second opening 316 via one or more of a spring arm, a ball spring, or other retaining mechanism.

Turning now to FIG. 4, it shows a detailed view 400 of the connection device 130 coupled to the first device 110 and the second device 120. The second body 224 is exposed and the first body and the third body of the pin 220 are inserted into the quick connect 310.

The clamp 210 is coupled to the body 122. In one example, the body 122 may be forced through the plurality of bristles 216 and enter the slot 218. The clamp 210 may be rotated such that the longitudinal axis 299 of the slot 218 may be parallel to a direction of travel 490. The direction of travel 490 may indicate a direction in which the first device 110 is pushed and the second device 120 rolls. The second device 120 may be moveable within the slot 218. The second device 120 may move along the longitudinal axis 299 and/or rotated within the slot 218.

In one example, the second device 120 may be positioned within the slot 218 and pressed against one of the first arm 211 of the second arm 213. In one example, the second device 120 may be pressed against the first arm 211 when travelling in a first direction, identical to the direction of travel 490, and pressed against the second arm 213 when travelling in a second direction, opposite the direction of travel 490.

Turning now to FIG. 5A, it shows a stored position 500 of the connection device 130. The connection device 130 may be stored on the first device 110. The pin (e.g., pin 220 of FIG. 2A) may be coupled to the quick connect 310 and the clamp 210 may be arranged on a hook, a pin, or other device. As illustrated, the connection device 130 may extend from a rear side 502 of the first device 110 to a mid-section of the first device 110, the mid-section between the rear side 502 and a front side 504. In one example, the clamp 210 is stored at the mid-section and the quick connect 310 is mounted adjacent to the rear side 502.

In one example, a stiffness of the arm 230 may include where the arm 230 is configured to maintain a position previously defined by a user. For example, if the arm 230 is positioned in the stored position 500, then the arm 230 may maintain the connection device 130 in the stored position 500 until a further force, not including gravity, is applied to the arm 230. Similarly, the arm 230 may maintain connected positions, as shown and described in FIGS. 5B and 5C until an additional force is applied to the arm 230. In this way, a position of the arm 230 does not change without an input. Additionally or alternatively, the connection device 130 may be wound, retracted, or stored in another way when in the stored position.

Turning now to FIG. 5B, it shows a first connection position 525. The connection device 130 is coupled to the quick connect 310 arranged at the rear side 502 of the first device 110. The clamp 210 of the connection device 130 extends away from the first device 110 and couples to the second device 120.

Turning now to FIG. 5C, it shows a second connection position 550. The second connection position 550 may differ from the first connection position 525 in that the pin of the connection device 130 is coupled to the quick connect 310 arranged at the front side 504 of the first device 110. As such, the first device 110 may include multiple mounting points for multiple quick connects. By doing this, multiple connection devices may be used to transport multiple other devices with the first device 110.

In some examples, additionally or alternatively, the quick connect 310 may be mounted to a track coupled to the first device 110. The quick connect 310 may be slidingly and fixedly coupled to the track based on an actuation of a clamp or other locking mechanism. For example, the quick connect 310 may be coupled to and moveable along the track when in a disengaged position. A user may move the quick connect 310 along the track to a determined position prior to locking the quick connect 310 in an engaged position. When in the engaged position, the quick connect 310 may not move along the track. The track may extend along an entire perimeter of the first device 110 or along an entire length of a side of the first device 110.

The disclosure provides support for a system including a first device, a second device, and a connection device coupled to a coupling element mounted on the first device at a first end and clamped to the second device at a second end, wherein the connection device further comprises a tether extending from the first end to the second end. A first example of the system further includes where the connection device comprises a pin at the first end and a clamp at the second end, the first end opposite the second end. A second example of the system, optionally including the first example, further includes where the tether comprises at least one wire arranged within an outer layer. A third example of the system, optionally including one or more of the previous examples, further includes where the tether comprises a fixed length along its longitudinal axis. A fourth example of the system, optionally including one or more of the previous examples, further includes where the tether is flexible about its radius. A fifth example of the system, optionally including one or more of the previous examples, further includes where the connection device comprises a clamp, wherein the clamp comprises a slot and a plurality of bristles arranged in an opening. A sixth example of the system, optionally including one or more of the previous examples, further includes where a length of the slot measured along its longitudinal axis is longer than a length of the opening. A seventh example of the system, optionally including one or more of the previous examples, further includes where the clamp is rotatable about the second device. An eighth example of the system, optionally including one or more of the previous examples, further includes where the second device is arranged within and moveable in the slot.

The disclosure provides additional support for a connection device including a clamp configured to couple to a tube, wherein the clamp comprises a U-shaped body, an opening, and a slot and a plurality of bristles extending between a first arm and a second arm of the clamp, wherein the plurality of bristles moves in response to a threshold force. A first example of the connection device further includes where the clamp is arranged at a first end of the connection device, further comprising a pin arranged at a second end of the connection device opposite the first end, wherein the pin engages with a coupling element spaced away from the tube. A second example of the connection device, optionally including the first example, further includes where the tube is a pole of an intravenous fluid (IV) pole. A third example of the connection device, optionally including one or more of the previous examples, further includes a tether extending from the clamp to a pin, wherein the tether comprising a at least one wire housed within an outer layer. A fourth example of the connection device, optionally including one or more of the previous examples, further includes where the plurality of wires is flexible in a radial direction of the tether. A fifth example of the connection device, optionally including one or more of the previous examples, further includes where a size of the slot is greater than a size of the opening.

The disclosure provides further support for a connection device configured to couple to a first device and a second device, the connection device including a clamp configured to couple to a pole of the second device, wherein the clamp comprises a U-shaped body, an opening, and a slot, a plurality of bristles extending between a first arm and a second arm of the clamp, wherein the plurality of bristles moves in response to a threshold force, and a pin arranged at an opposite end of the connection device relative to the clamp, wherein the pin engages with a coupling element of the first device. A first example of the connection device further includes where the connection device is stored on the first device. A second example of the connection device, optionally including the first example, further includes where the coupling element is one of a plurality of coupling elements mounted to the first device. A third example of the connection device, optionally including one or more of the previous examples, further includes where the first device is a bed frame and the second device is an intravenous fluid (IV) pole. A fourth example of the connection device, optionally including one or more of the previous examples, further includes where a flexible tether extends between the clamp and the pin.

As used herein, the term “approximately” is construed to mean plus or minus five percent of the range unless otherwise specified.

As used herein, an element or step recited in the singular and preceded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A system, comprising:
a first device;
a second device; and
a connection device coupled to a coupling element mounted on the first device at a first end and clamped to the second device at a second end, wherein the connection device further comprises a tether extending from the first end to the second end.

2. The system of claim 1, wherein the connection device comprises a pin at the first end and a clamp at the second end, the first end opposite the second end.

3. The system of claim 1, wherein the tether comprises at least one wire arranged within an outer layer.

4. The system of claim 1, wherein the tether comprises a fixed length along its longitudinal axis.

5. The system of claim 1, wherein the tether is flexible about its radius.

6. The system of claim 1, wherein the connection device comprises a clamp, wherein the clamp comprises a slot and a plurality of bristles arranged in an opening.

7. The system of claim 6, wherein a length of the slot measured along its longitudinal axis is longer than a length of the opening.

8. The system of claim 6, wherein the clamp is rotatable about the second device.

9. The system of claim 6, wherein the second device is arranged within and moveable in the slot.

10. A connection device, comprising:
a clamp configured to couple to a tube, wherein the clamp comprises a U-shaped body, an opening, and a slot; and
a plurality of bristles extending between a first arm and a second arm of the clamp, wherein the plurality of bristles moves in response to a threshold force.

11. The connection device of claim 10, wherein the clamp is arranged at a first end of the connection device, further comprising a pin arranged at a second end of the connection device opposite the first end, wherein the pin engages with a coupling element spaced away from the tube.

12. The connection device of claim 10, wherein the tube is a pole of an intravenous fluid (IV) pole.

13. The connection device of claim 10, further comprising a tether extending from the clamp to a pin, wherein the tether comprising a at least one wire housed within an outer layer.

14. The connection device of claim 13, wherein the plurality of wires is flexible in a radial direction of the tether.

15. The connection device of claim 10, wherein a size of the slot is greater than a size of the opening.

16. A connection device configured to couple to a first device and a second device, the connection device comprising:
a clamp configured to couple to a pole of the second device, wherein the clamp comprises a U-shaped body, an opening, and a slot;
a plurality of bristles extending between a first arm and a second arm of the clamp, wherein the plurality of bristles moves in response to a threshold force; and
a pin arranged at an opposite end of the connection device relative to the clamp, wherein the pin engages with a coupling element of the first device.

17. The connection device of claim 16, wherein the connection device is stored on the first device.

18. The connection device of claim 16, wherein the coupling element is one of a plurality of coupling elements mounted to the first device.

19. The connection device of claim 16, wherein the first device is a bed frame and the second device is an intravenous fluid (IV) pole.

20. The connection device of claim 16, wherein a flexible tether extends between the clamp and the pin.
